(19) United States
(12) Patent Application Publication
(10) Pub. No.: US 2016/0175135 A1
(43) Pub. Date: Jun. 23, 2016

(54) BRACE WITH INCLINATION ADJUSTMENT

(71) Applicant: Albrecht GmbH, Stephanskirchen (DE)

(72) Inventors: Martin Kahles, Halfing (DE); Hans-Georg Opahle, Rosenheim (DE)

(21) Appl. No.: 14/978,940

(22) Filed: Dec. 22, 2015

(30) Foreign Application Priority Data
Dec. 23, 2014 (DE) 102014019715.8

(51) Int. Cl. A61F 5/01

(52) U.S. Cl. CPC A61F 5/0125

(57) ABSTRACT

An orthotic, in particular a knee orthotic, comprises a pair of rail arms, which in each case have a pivoting first rail arm section and a second rail arm section that can be fastened to a patient's body. The second rail arm section of at least one rail arm is held on the first rail arm section in a slope—adjustable manner and in a movable manner in the longitudinal direction of the rail arm and is forcibly controlled such that a pivoting of the rail arms in the flexion or extension direction is coupled both with an adjustment in the medial or lateral inclination of the second rail arm section and with a change in length of the rail arm.

This application claims priority to German Patent Application No. 102014019715.8 filed Dec. 23, 2014, the entire contents of which is hereby incorporated by reference.

### BACKGROUND OF THE INVENTION

The invention relates to an orthotic, in particular a knee orthotic, with a pair of rail arms, which in each case have a pivoting first rail arm section and a second rail arm section that can be fastened to a patient's body, an articulated system for pivoting the rail arm in the flexion and extension directions, and an inclination adjustment system for adjusting the medial or lateral inclination of the second rail arm section of at least one rail arm relative to the articulated system, according to the preamble of claim 1.

Orthotics of this type are used in particular in patients who suffer from a medial or lateral knee joint arthrosis (gonarthrosis). In the case of a medial knee joint arthrosis, the medial (inner) compartment of the femorotibial joint is affected, while in the case of a lateral knee joint arthrosis, an arthrosis of the lateral (outer) femorotibial compartment exists. A medial knee joint arthrosis occurs in particular in individuals who have a bowleg joint misalignment. In this case, it is a varus gonarthrosis. A lateral knee joint arthrosis frequently occurs in patients who have a knock-knee misalignment. In this case, it is a valgus gonarthrosis.

In patients with a medial or lateral gonarthrosis, in many cases, the use of orthotics supplementing medical, physiotherapeutic and/or operative therapies is advantageous. In many cases, it is possible to use suitable orthotics to alleviate the patient's activity-related pain and to improve the joint function. In addition, patients with knee joints that are completely destroyed arthrotically and a varus or valgus instability can be restored to limited ambulatory status by such orthotics. In a non-operative manner, such orthotics offer the possibility of exerting a valgus or varus stress externally and thus of relieving the load of the affected compartment. In addition, such orthotics also have proven their value post-operatively after operations for building up joint cartilage or meniscus reconstruction. Also preoperatively, before a corrective valgisation osteotomy of the knee joint, the success of the treatment that is to be expected can be simulated by such orthotics to a certain extent.

In order to exert a medially or laterally directed force on the knee joint, an orthotic is already known from WO 94/15555 A1, in which the pivotable rail arms are divided into two in each case. There, the rail arms in each case have a first rail arm section, which is mounted to pivot on a carrier plate of the rail joint, and a second rail arm section, which can be fastened on the first rail arm section by means of adjusting screws in a certain medial or lateral angular position and on the patient's upper or lower leg by means of cuffs. In this case, it is disadvantageous that after the adjustment of the medial or lateral inclination (tilt) of the second rail arm section, the medial or lateral force acts incessantly on the knee joint or the upper and lower leg, which is regarded as unpleasant by the patients and can even result in undesirable pressure points, traumas and circulatory disorders at those points at which the rail arms are fastened on the body. Furthermore, the desired load relief of the damaged knee joint compartment is not provided to the desired extent by the orthotic located there.

From U.S. Pat. No. 7,500,957 B2, an orthotic according to the preamble of claim 1 is known, in which the outer rail arm sections are mounted to pivot in the medial or lateral direction on inner rail arm sections by means of additional joints. The outer rail arm sections are actively inclined there in the direction of the leg by means of a pin arrangement when the knee is moved into its full extension position. It has been shown, however, that even by this orthotic, the desired effect of load relief for the knee joint is not achieved.

### SUMMARY OF THE INVENTION

An object of the described embodiments is therefore to provide an orthotic of the above-mentioned type, which is especially suitable for medial and lateral arthroses in the medical and ergonomic respect and makes possible an especially effective load relief of the damaged joint compartment.

In the case of the orthotic according to the described embodiments, the second rail arm section of at least one rail arm that can be fastened to the patient's body is held on the first rail arm section in a slope—adjustable manner and in a movable manner in the longitudinal direction of the rail arm and is forcibly controlled such that a pivoting of the rail arm in the flexion or extension direction is coupled both with an adjustment of the medial or lateral inclination of the second rail arm section and with a change in length of the rail arm.

In contrast to the known orthotics, in which the medial or lateral inclination of the second rail arm section remains the same over the entire pivoting area of the rail arm, the orthotic according to the described embodiments makes possible a change in the inclination based on the pivoting position of the rail arm. Thus, it is possible in particular to minimize the inclination when the joint, in particular the knee joint, is greatly angled, for example when the lower leg and upper leg occupy an angle to one another of 90° or less, as is the case when sitting. In this greatly angled state of the knee joint and thus also the two rail arms, even in the case of a greater inclination of the rail arms, the medial or lateral forces exerted by the rail arms would not act on the knee joint in the desired manner, so that the minimization of the inclination in the greatly angled position of the rail arm does not have any negative effect with respect to the load relief of the damaged knee joint compartment. The desired load relief of the damaged knee joint compartment can, however, be brought about especially effectively when the knee is extended or is located at least close to its extended position. Using the orthotic according to the described embodiments, it is therefore possible to reduce the medial and lateral compressive and tensile forces of the orthotic in the angled position of the knee joint at least to a large extent, while they are applied to the patient's body in the position of the knee extended to its full extent. This leads to a significantly improved wearing comfort and prevents traumas and circulatory disorders in those areas of the body on which the rail arms rest or to which they are fastened. In particular, load is removed from the patient when the patient occupies a sitting position.

In addition, it is of special advantage that in the case of a pivoting of the orthotic in the flexion or extension direction, not only is an active, dynamic slope adjustment carried out in the medial or lateral direction, but rather at the same time, a predetermined lengthwise adjustment of the rail arm is also carried out. The orthotic according to the described embodiments is designed in particular in such a way that when a knee is moved into its full extension position, the second rail arm section is removed from the pivoting axis that is assigned to the rail arm in question and thus removed from the central articulated system, so that the rail arm is extended. Thus, the knee joint is actively pulled apart via the cuffs fastened to the upper and lower leg. This results in an especially effective load relief of the damaged knee joint compartment and prompts the corrective movement in the medial or lateral direction that is applied on the knee joint by the medial or lateral inclination of the rail arm. If the knee is bent, however, the rail arms are advantageously shortened in the case of simultaneous reduction of the medial or lateral inclination.

According to an advantageous embodiment, the inclination adjustment system comprises a slotted guide, with which the second rail arm section is guided in a movable manner on the first rail arm section. In this case, the slotted guide can comprise at least one guide link that is arranged on the first or second rail arm section, bent in medial or lateral direction and/or oblique, and at least one guide element that is arranged on the other rail arm section and engaging in the guide link. Such slotted guides make it possible in particular to change the inclination continuously and uniformly when the rail arms are pivoted in the extension or flexion direction, while at the same time, a stable and precise guiding of the second rail arm section on the first rail arm section is ensured.

Advantageously, the first or second rail arm section has a U-shaped end section with opposing guide legs, in which in each case, a guide link is arranged, whereby the other thus interacting rail arm section has a guide section, insertable between the guide legs, with guide elements arranged on opposite sides of the guide section. Thus, an especially stable and exact guiding of the second rail arm section on the first rail arm section can be achieved.

Advantageously, the guide element that engages in the guide link is designed as an elongated guiding ridge that is matched to the shape of the guide link. As an alternative to this, however, other types of guide elements are also conceivable, for example guide bolts or pins that are at a distance from one another.

According to an advantageous embodiment, the articulated system has at least one carrier plate, on which the first rail arm section is mounted to pivot around at least one swivel axis, whereby a shifting system is provided that is in operative connection, on the one hand, with the carrier plate and, on the other hand, with the second rail arm section of at least one rail arm such that a pivoting of the rail arm in the flexion or extension direction is forcibly coupled from or to the swivel axis both with an adjustment of the second rail arm section in the medial or lateral direction and with the second rail arm section moving away or moving closer. Thus, in a simple and reliable way, the second rail arm section can be moved relative to the first rail arm section and thus to the articulated system such that the inclination and the longitudinal position relative to the central articulated system changes in the desired way.

Advantageously, the shifting system comprises at least one connecting brace, which is connected, on the one hand, at a distance from the swivel axis with the carrier plate and, on the other hand, with the second rail arm section of at least one rail arm, such that when the rail arm pivots in the flexion or extension direction, the second rail arm section shifts relative to the first rail arm section.

### BRIEF DESCRIPTION OF THE DRAWINGS

The described embodiments will be explained in more detail below based on drawings by way of example. Here:

FIG. 1 shows an orthotic applied on the upper and lower leg for load relief of a knee joint,

FIG. 2a shows a right leg with a bowleg joint misalignment,

FIG. 2b shows a diagrammatic view of the orthotic of FIG. 1 with medial arrangement on the leg of FIG. 2a after correction of the bowleg joint misalignment,

FIG. 2c shows a right leg with a knock-knee joint misalignment,

FIG. 2d shows a diagrammatic view of the orthotic of FIG. 1 with lateral arrangement on the leg of FIG. 2c after correction of the knock-knee joint misalignment,

FIG. 3 shows an exploded view of the orthotic,

FIG. 4 shows a spatial view of the first rail arm section obliquely from above,

FIG. 5 shows a lengthwise section through the orthotic, whereby the rail arms are depicted shortened and are located at an angle of 180° to one another,

FIG. 6 shows a side view of the orthotic of FIG. 5, whereby an outer carrier plate for illustrating the subjacent parts is not depicted,

FIG. 7 shows a front view of the orthotic of FIGS. 5 and 6,

FIG. 8 shows a side view of the orthotic, whereby an outer carrier plate for illustrating the subjacent parts is not depicted,

FIG. 9 shows a front view of the angled orthotic of FIG. 8, and

FIG. 10 shows a front view of the extended orthotic of FIG. 7 for illustrating the change in inclination compared to FIG. 9.

### DETAILED DESCRIPTION OF THE INVENTION

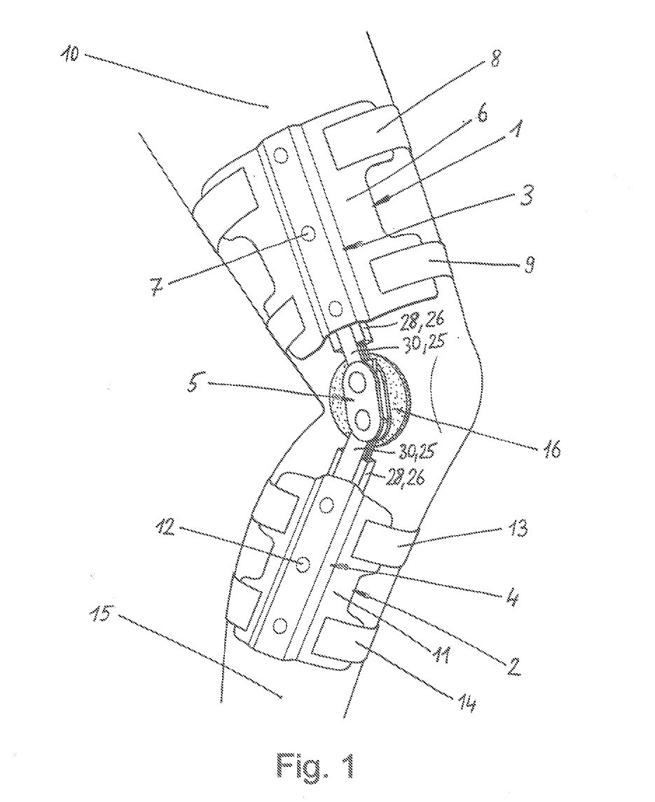

Based on FIGS. 1 to 10, an embodiment of the orthotic in the form of a knee orthotic is described in more detail below. The orthotic according to the described embodiments can, however, also be used for other joints, for example the elbow joint.

FIG. 1 shows an orthotic with an upper cuff 1, a lower cuff 2, a first rail arm 3, a second rail arm 4, and a central articulated system 5 for pivotable connection of the first and second rail arms 3, 4.

The upper cuff 1 has an upper cuff shell 6, to which the first rail arm 3 is fastened by means of suitable fastening agents, for example by means of rivets 7. A proximal holding band 8 and a distal holding band 9 for fastening the upper cuff 1 and thus the first rail arm 3 to a patient's upper leg 10 are fastened to the upper cuff shell 6.

The lower cuff 2 is designed in a similar manner and has a lower cuff shell 11, to which the second rail arm 4 is fastened by means of suitable fastening agents, for example by means of rivets 12, as well as a proximal holding band 13 and a distal holding band, with which the lower cuff shell 11 and thus the second rail arm 4 can be fastened to a patient's lower leg 15.

The orthotic should have appropriate padding in order to improve wearing comfort. Of this padding, however, only a cushion 16 that is located on the side of the articulated system 5 that faces the leg is depicted.

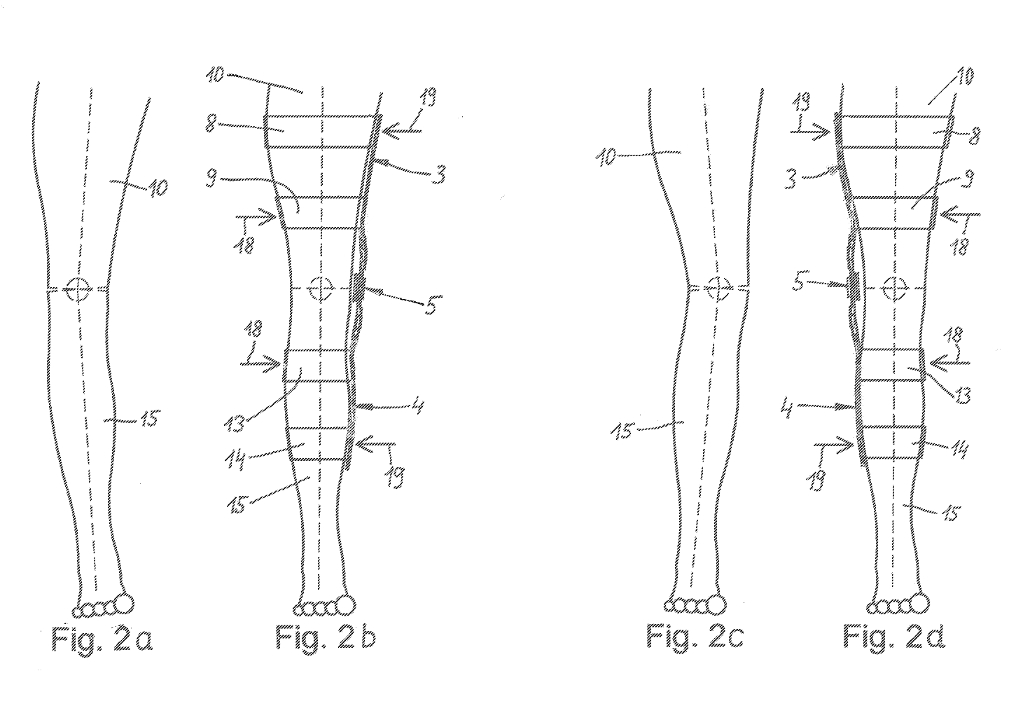

As can be seen from FIGS. 2a to 2d, which show a view of a leg from the front, the orthotic can be arranged on the medial side of the leg (FIG. 2b) or on the lateral side of the leg (FIG. 2d). The medial arrangement according to FIG. 2b is applied when there is a bowleg, i.e., when load is to be removed from the medial compartment of the knee. The lateral arrangement of the orthotic according to FIG. 2d is applied when there is a knock-knee, and load is to be removed from the lateral compartment of the knee joint.

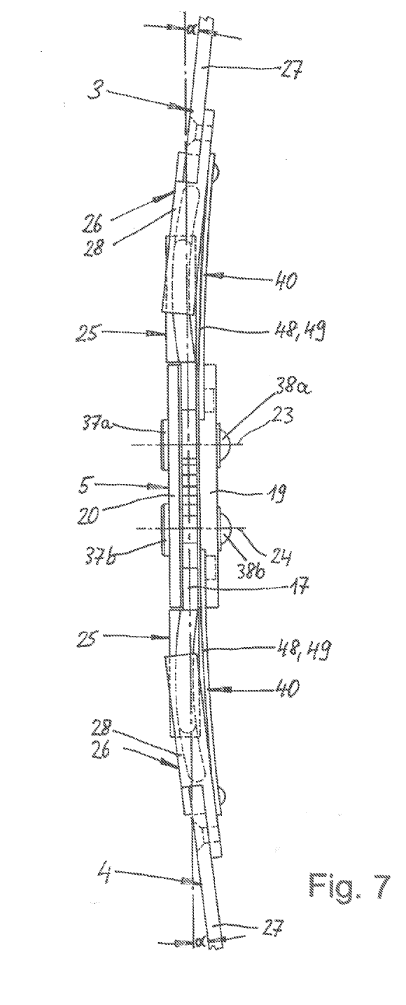

FIGS. 7 and 10 show that the first rail arm 3 and second rail arm 4 in the extension position can be sloped by a certain inclination angle α in the medial or lateral direction relative to a main plane 17 of the articulated system 5. The proximal end of the first (upper) rail arm 3 as well as the distal end of the second (lower) rail arm 4 in this case are at a greater distance from the main plane 17 than the distal end of the first rail arm 3 or the proximal end of the second rail arm 4. In an arrangement of the orthotic according to FIGS. 2b and 2d, the proximal end of the first rail arm 3 rests on the upper leg 10 in the area of the proximal holding band 8, while the first rail arm 3 is a certain distance from the upper leg 10 in the area of the distal holding band 9 at least until the distal holding band 9 is tightened. In the same way, the distal end of the second (lower) rail arm 4 rests on the lower leg 15 in the area of the distal holding belt 14, while the second rail arm 4 is a certain distance from the lower leg 15 in the area of the proximal holding band 13 at least until the proximal holding band 13 is tightened. If the holding bands 9, 13 that are closer to the knee joint are now tightened, forces are applied on the upper leg 10 and the lower leg 15 in the direction of the arrow 18, and opposing forces are applied in the area of the holding bands 8, 14 in the direction of the arrow 19, which forces push out the knee joint in the medial direction (FIG. 2b) or lateral direction (FIG. 2d). This produces a corresponding load relief of the medial knee joint compartment (FIG. 2b) or lateral knee joint compartment (FIG. 2d).

The medial or lateral inclination angle α, depicted in FIGS. 7 and 10, i.e., the angular positions of the first and second rail arms 3, 4 in the area of the holding bands 8, 9, 13, 14 relative to the main plane 17 of the articulated system 5, suitably exists only when the leg is extended, i.e., when the first and second rail arms 3, 4 are at an angle to one another of 180° or close to 180°, in particular 160° to 180°. This inclination angle α, which also can be referred to as the first inclination angle, can be, for example, 2° to 10°, preferably 3° to 7°. Other first inclination angles α are possible. If the knee is bent, i.e., the orthotic is pivoted in the flexion direction, the inclination angle α at the same time is changed to a second inclination angle, which is different from the first inclination angle and can be, for example, 0° to 2°, in particular 0°, relative to the main plane 17. This second inclination angle preferably is adjusted when the knee is greatly bent, i.e., when the angle between the first and second rail arms 3, 4 is 90° or less. In this bent state, the medial and lateral tensile and compressive forces exerted on the leg are considerably reduced by the orthotic via the holding bands 8, 9, 13, 14, by which the blood supply to the leg is conveyed into these areas and the wearing comfort is considerably improved.

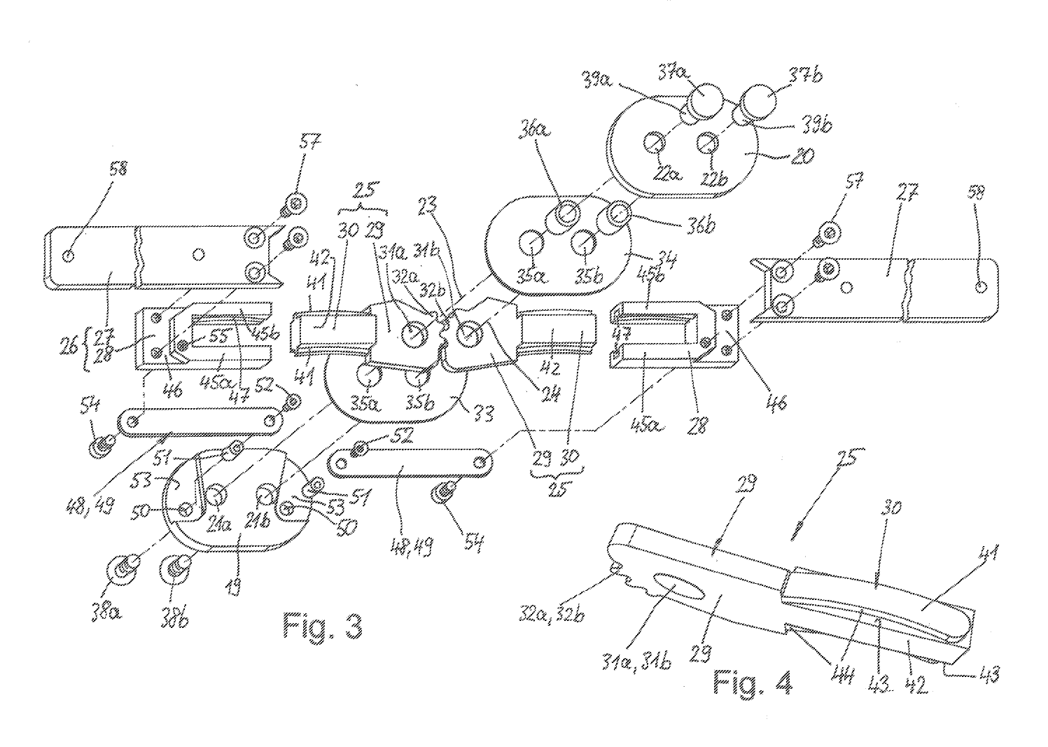

The structure of the orthotic according to the described embodiments, which makes possible the automatic change of the inclination angle α with the simultaneous change in length of the rail arms 3, 4 based on the flexion or extension angle of the rail arms 3, 4, is described in more detail below based on FIGS. 3 to 8. The cuffs 1, 2 as well as the cushion materials are omitted in these figures for the sake of clarity.

FIG. 3 shows the orthotic in exploded view. The articulated system 5 for pivoting the two rail arms 3, 4 comprises a first carrier plate 19 and a second carrier plate 20 that is parallel thereto. Both carrier plates 19, 20 are designed in an oval shape and have the same peripheral contour. Holes 21a, 21b or 22a, 22b are provided in the carrier plates 19, 20. The holes 21a, 22a are aligned with one another and have a common center axis, which forms a first swivel axis 23 for the first rail arm 3. The holes 21b, 22b are also aligned with one another and have a common center axis, which forms a second swivel axis 24 for the second rail arm 4. The two swivel axes 23, 24 are parallel to one another. The carrier plates 19, 20 are thus part of the central articulated system 5, with which the rail arms 3, 4, are mounted to pivot biaxially in order to be able to be pivoted in the flexion or extension direction.

The rail arms 3, 4 in each case comprise a first rail arm section 25, which is mounted to pivot on the carrier plates 19, 20, as well as a second rail arm section 26, which comprises a rail fastening section 27 and an end section 28 and is mounted to move longitudinally on the first rail arm section 25 by means of the end section 28.

As can be seen in particular from FIG. 3, the end sections 28 of the second rail arm section 26 are designed separately from the assigned rail fastening sections 27 and are tightly secured to the assigned rail fastening sections 27 by means of screws 57. As an alternative to this, it is also possible, however, to design end sections 28 and rail fastening sections 27 in one piece.

The rail fastening sections 27 have holes 58, which are used to guide the fastening agents, in particular the rivets 7, 12, with which the rail fastening sections 27 are fastened to the cuff shells 6, 11.

The first rail arm sections 25 in each case comprise a plate-shaped bearing section 29 and a guide section 30 that extends behind it in the direction of the second rail arm section 26. In the embodiment shown, bearing section 29 and guide section 30 are designed in one piece. As an alternative, however, it is also possible to design the bearing section 29 and the guide section 30 in two pieces and to connect them securely to one another by means of suitable fastening agents.

The plate-like bearing section 29 of each first rail arm section 25 has a hole 31a or 31b, which is aligned with the swivel axis 23 or 24. The pivoting angles of the first rail arm section 25 relative to the carrier plates 19, 20 are matched to one another by gears 32a, 32b, which are provided on opposite front surfaces of the bearing section 29 and engage in one another.

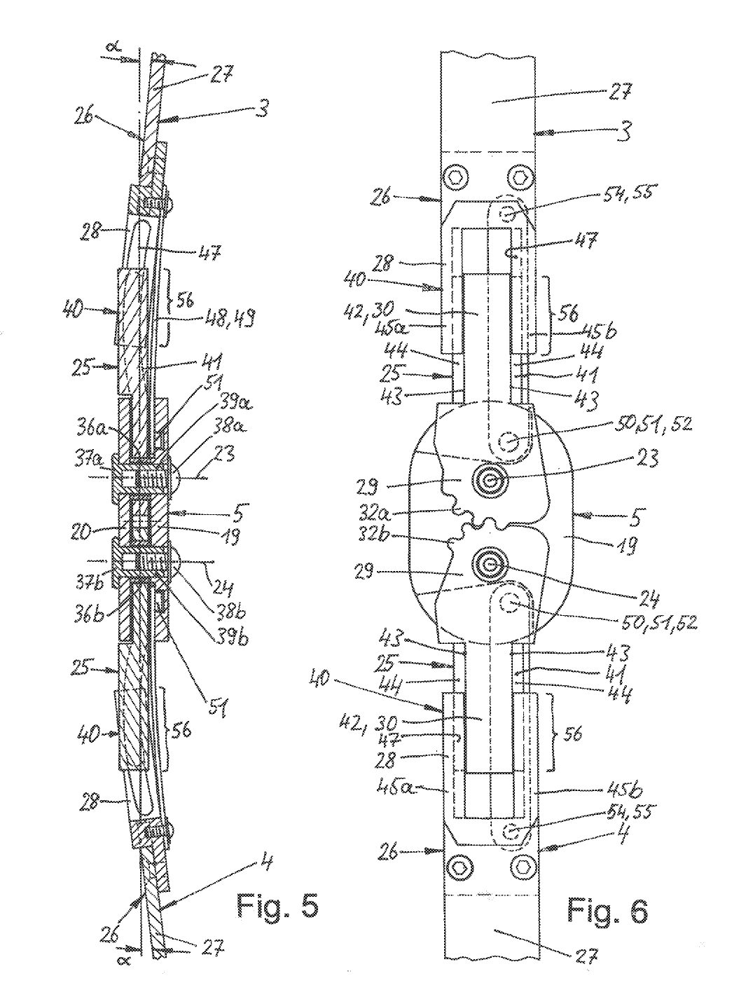

As can also be seen from FIGS. 5 and 6, the thickness of the plate-like bearing sections 29 are proportioned in such a way that they are arranged essentially with zero play in the intermediate space between the carrier plates 19, 20 that are arranged in parallel. The guide sections 30 are located, however, outside of the carrier plates 19, 20 and can therefore, as shown, have a larger thickness than the bearing sections 29.

To reduce friction, friction-reducing disks 33, 34 are provided on both sides of the bearing sections 29 between the latter and the carrier plates 19, 20, which disks have the same oval contour as the carrier plates 19, 20 in the embodiment that is shown. In the friction-reducing disks 33, 34, in turn two holes 35a, 35b are arranged in each case aligned with the swivel axes 23, 24. The material of the friction-reducing disks 33, 34 is preferably a friction-free plastic, for example Teflon.

The mounting of the first rail arm section 25 on the carrier plates 19, 20 is done by means of cylindrical inner bearing sleeves 36a, 36b as well as outer threaded sleeves 37a, 37b and screws 38a, 38b. The bearing sleeves 36a, 36b extend through the holes 35a, 31a or 35b, 31b of the friction-reducing disks 33, 34 and bearing sections 29. The outside diameter of the bearing sleeves 36a, 36b is in this case matched to the diameter of these holes such that a very exact radial guiding of the bearing section 29 is provided.

The length of the bearing sleeves 36a, 36b determines the distance between the carrier plates 19, 20.

The threaded sleeves 37a, 37b in each case have a hollow cylindrical section 39a, 39b with a reduced outside diameter as well as a larger head section. The hollow cylindrical sections 39a, 39b extend essentially with zero play through the holes 22a, 22b of the second carrier plate 20, the inner through-holes of the bearing sleeves 36a, 36b, as well as at least partially through the holes 21a, 21b of the first carrier plate 19, as can also be seen from FIG. 5. In addition, the hollow-cylindrical sections 39a, 39b are provided with an inside threading, in which the screws 38a, 38b can be screwed in from the opposite side of the articulated system 5 such that the two carrier plates 19, 20 are held securely to one another. In this case, as already explained, the bearing sleeves 36a, 36b are used as spacers for the carrier plates 19, 20.

By such a design, it is possible to connect the first rail arm sections 25 of the rail arms 3, 4 in a pivotable manner to one another such that a pivoting in the flexion and extension directions of the first rail arm section 25 is possible within a prescribed pivoting area, while they can move little or not at all in the medial and lateral directions through the carrier plates 19, 20 and friction-reducing disks 33, 34.

The medial or lateral pivoting of the second rail arm section 26 relative to the first rail arm section 25 is carried out by means of an inclination adjustment system 40, which acts between the end section 28 of the second rail arm sections 26 and the guide section 30 of the first rail arm sections 25 and is forcibly coupled with the articulated system 5 such that a pivoting of the first and second rail arms 3, 4 in the flexion or extension direction is connected to a change of the inclination angle α in the medial or lateral direction relative to the main plane 17 of the articulated system 5. In this case, a plane in which the bearing sections 29 of the first rail arm sections 25 lie and that is thus parallel to the main planes of the carrier plates 19, 20 is defined as main plane 17.

In the depicted embodiment, the inclination adjustment system 40 comprises a slotted guide with which the second rail arm section 26 of each rail arm 3, 4 is guided onto the first rail arm section 25 to be able to move in its longitudinal direction.

In this connection, the guide sections 30 of the first rail arm sections 25 in each case have two opposite guiding ridges 41, which run in the longitudinal direction of the guide sections 30, and, as can be seen in particular from FIG. 4, are designed in a bent manner in the medial or lateral direction. The curve thus lies in a plane that is arranged at right angles to the main plane 17 of the first rail arm section 25. The guiding ridges 41 have a constant height by which they project on opposite sides of a parallelepiped-shaped extension 42 of the bearing sections 29 via flat base surfaces 43 of the extension 42. Thus, side guiding surfaces 44 that are bent in the shape of a circular arc on the guiding ridges 41 are formed.

On their end sections 28, the second rail arm sections 26 have slotted guides that are designed according to the shape of the guiding ridges 41, so that the end sections 28 and thus the entire second rail arm sections 26 are guided through the flat base surfaces 43 and the bent side guiding surfaces 44 of the guiding ridges 41. In this connection, the end sections 28 are designed U-shaped and have opposing guide legs 45a, 45b, which extend parallel to one another from a connecting section 46. In each case, a bent guide link 47, in which a guiding ridge 41 of the first rail arm section 25 is guided to move longitudinally, is located in the guide legs 45a, 45b. In addition, the inside surfaces of the guide legs 45a, 45b rest on the base surfaces 43 of the extension 42. If the end pieces 28 are moved along the guide section 30 of the first rail arm sections 25, the end piece 28 and thus the entire second rail arm section 26 execute an arched movement curve, by which the second rail arm section 26 is pivoted in medial or lateral direction relative to the first rail arm section 25.

The lengthwise shifting of the second rail arm section 26 relative to the first rail arm section 25 is carried out by means of a shifting system 48, which comprises a connecting element in the form of a connecting brace 49. This connecting brace 49 is fastened to a first end on the first carrier plate 19 of the articulated system 5. In this connection, the first carrier plate 19, as can be seen from FIG. 3, has a hole 50, in which a sleeve 51 with inside threading is mounted. The connecting brace 49 can be fastened to this sleeve 51 by means of a screw 52, which is guided through a corresponding hole of the connecting brace 49 and is screwed into the inside threading of the sleeve 51. The sleeve 51 is mounted to rotate in the hole 50 so that the connecting brace 49 can be pivoted relative to the carrier plate 19.

So that the connecting brace 49 has room between the two carrier plates 19, 20, the carrier plate 19 has two recessed pockets, i.e., recesses 53, which extend over the optional pivoting area of the connecting braces 49. The connecting braces 49 extend into the area of these recesses 43 such that they project crosswise to the carrier plate 19, not over the inner side surface of the carrier plate 19.

In addition, the connecting braces 49 are coupled in a pivotable manner in their second opposing end area with the second rail arm sections 26. This is done with screws 54, which are guided through a hole of the connecting braces 49 and are screwed into a threaded hole 55 of the end sections 28 of the second rail arm sections 26.

Hole 50, sleeve 51 and screw 52 thus form a first or inner bearing point for coupling the connecting braces 49 to the articulated system 5, while screw 54 and threaded hole 55 form a second or outer bearing point for coupling the connecting braces 49 with the second rail arm sections 26. In this case, the first bearing point is arranged offset to the respective swivel axes 23, 24 such that when the rail arms 3, 4 are moved from their maximally extended position (extension position) into their maximally angled position (flexion position), the second or outer bearing point, because of the connecting braces 49, describes a circle that continuously approaches the swivel axes 23 or 24. This means that the second rail arm sections 26 are continuously shifted in the direction of the carrier plates 19, 20 in the case of a movement from the extension position into the flexion position by means of the connecting braces 49, by which the sliding arms 3, 4 are shortened. In this case, the end sections 28 glide along the guide sections 30 of the first rail arm sections 25 and are simultaneously pivoted in the medial or lateral direction in this case corresponding to the arched contour of the guide links 47 and guiding ridges 41.

Conversely, the second rail arm sections 26 are moved away in their longitudinal direction from the swivel axes 23 or 24 and thus from the bearing sections 29, i.e., removed, when the rail arms 3, 4 are pivoted from their maximal flexion position into their extension position. The sliding arms 3, 4 in this case are extended accordingly.

In the embodiment depicted, this lengthening of the rail arms 3, 4 brings about that the upper cuff 1 is moved in the proximal direction and the lower cuff 2 is moved in the distal direction, when the orthotic is moved from its flexion position into its extension position. Thus, the femorotibial compartment is pulled apart.

FIGS. 5 to 7 show the orthotic in its maximal extension position, in which the rail arms 3, 4 are arranged at an angle of 180° to one another in the side view that is shown in FIG. 6. As can be seen in particular from FIG. 6, the end sections 28 of the second rail arm sections 26 and the guiding sections 30 of the first rail arm sections 25 overlap in this position in an overlapping area 56. The latter is large enough to ensure an exact guiding of the second rail arm sections 26 to the first rail arm sections 25 in all directions. As can be seen from FIG. 5, the guide links 47 and guiding ridges 41 run into this overlapping area 56 such that the second rail arm sections 26 are obliquely sloped in the medial or lateral direction relative to the main plane 17 of the articulated system 5 and have an inclination angle α, which is approximately 5° in the embodiment that is shown.

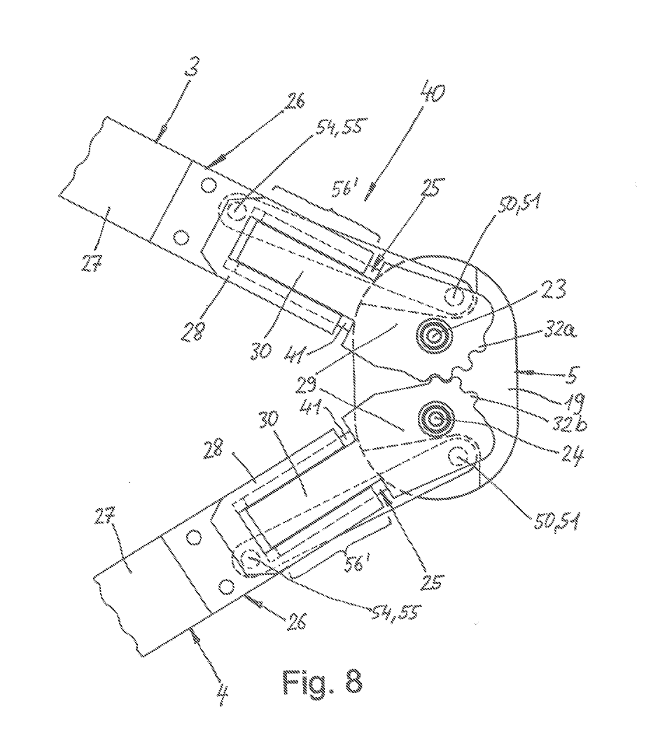

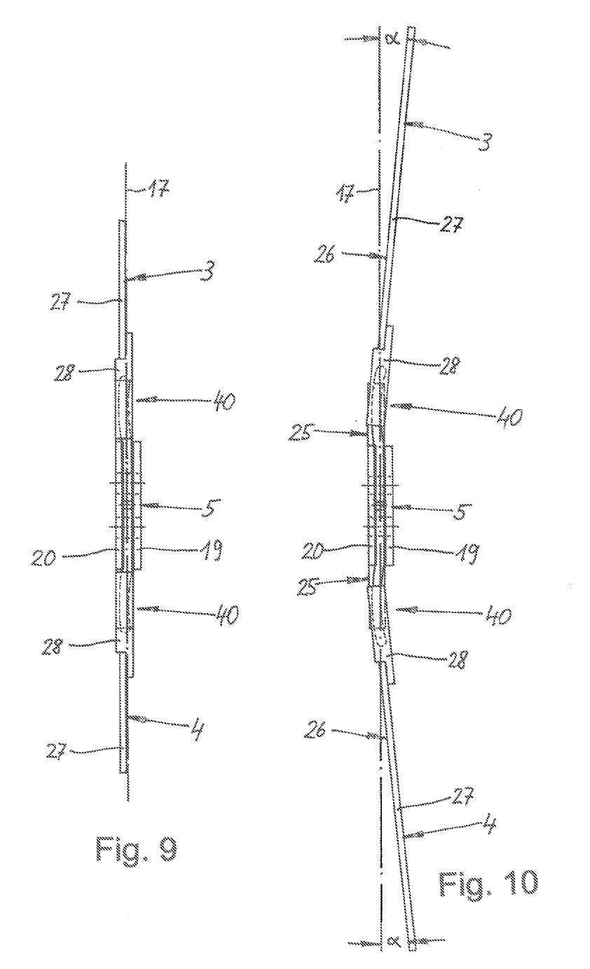

In FIGS. 8 and 9, a position of the orthotic is shown, in which the rail arms 3, 4 are maximally bent toward one another, i.e., occupy a minimal flexion angle with one another. This is approximately 60° in the embodiment that is shown. As can be seen, the second rail arm sections 26 are located closer to the respective bearing sections 29 or swivel axes 23, 24 in this position because of the positional control by the connecting braces 49. The overlapping area 56′ is correspondingly enlarged. Guide links 47 and guiding ridges 41 are designed such that in this position, the medial or lateral inclination angle α of the second rail arm sections 26 is reduced, and in this embodiment is 0°, as can be seen from FIG. 9.

Within the scope of the described embodiments, a number of variations are possible. For example, it is possible in principle to design the inclination adjustment system 40 and rail arm-length-changing system to be not the same on both rail arms 3, 4 but rather different, such that in the case of a change of the flexion or extension angle of the rail arms 3, 4, the medial or lateral inclination angle α and/or the length of the first rail arm 3 changes in a way that is unlike that of the second rail arm 4. In addition, it is also possible to provide the inclination adjustment system 40 and the rail arm-length-changing system only on one of the rail arms 3, 4. In addition, the described embodiments can be used not only in the case of orthotics with biaxial joints, but also in the case of those that have only a single swivel axis. The guide links 47 and guiding ridges 41 do not necessarily have to be curved in the shape of a circular arc, but rather can also have other curvatures. The end sections 28 can also be designed in one piece with the rail fastening sections 27. While, according to FIGS. 2a and 2b, the orthotic according to the described embodiments is arranged only on the medial or lateral side of a body joint, it is also possible in addition to arrange an orthotic without an inclination adjustment system or an orthotic according to the described embodiments on the opposite side, such that the load-relief movement for the damaged joint compartment is supported. In addition, the guide links 47 can also be provided on the pivoting first rail arm sections 25, and the thus cooperating guide elements can be provided on the second rail arm sections 26.

1. Orthotic, in particular a knee orthotic, which comprises:
a pair of rail arms, which in each case have a pivoting first rail arm section and a second rail arm section that can be fastened to a patient's body,
an articulated system for pivoting the rail arms in the flexion and extension directions, and
an inclination adjustment system for adjusting the medial or lateral inclination of the second rail arm section of at least one rail arm relative to the articulated system,
whereby the second rail arm section of at least one rail arm is guided in a movable manner to the first rail arm section and is coupled to the articulated system, such that a pivoting of the rail arms in flexion or extension direction is forcibly coupled with an adjustment of the medial or lateral inclination of the second rail arm section,
wherein the second rail arm section of at least one rail arm is held on the first rail arm section in a slope-adjustable manner and in a movable manner in the longitudinal direction of the rail arm and is forcibly controlled, such that a pivoting of the rail arm in the flexion or extension direction is coupled both with an adjustment of the medial or lateral inclination of the second rail arm section and with a change in length of the rail arm.

2. Orthotic according to claim 1, wherein the inclination adjustment system comprises a slotted guide with which the second rail arm section is guided in a movable manner on the first rail arm section.

3. Orthotic according to claim 2, wherein the slotted guide comprises at least one guide link that is arranged on the first or second rail arm section, bent in medial or lateral direction and/or oblique, and at least one guide element that is arranged on the other rail arm section and engaging in the guide link.

4. Orthotic according to claim 3, wherein the first or second rail arm section has a U-shaped end section with opposing guide legs, in which in each case, a guide link is arranged, and wherein the other rail arm section has a guide section, insertable between the guide legs, with guide elements arranged on opposite sides of the guide section.

5. Orthotic according to claim 3, wherein the guide element is designed as an elongated guiding ridge matched to the shape of the guide link.

6. Orthotic according to claim 1, wherein the articulated system has at least one carrier plate, on which the first rail arm section is mounted to pivot around at least one swivel axis, and wherein a shifting system is provided that is in operative connection, on the one hand, with the carrier plate, and, on the other hand, with the second rail arm section of at least one rail arm such that a pivoting of the rail arm in the flexion or extension direction is forcibly coupled from or to the swivel axis both with an adjustment of the second rail arm section in the medial or lateral direction and with the second rail arm section moving away or moving closer.

7. Orthotic according to claim 6, wherein the shifting system comprises at least one connecting brace, which is connected, on the one hand, at a distance from the swivel axis with the carrier plate and, on the other hand, with the second rail arm section of at least one rail arm, such that when the rail arm pivots in the flexion or extension direction, the second rail arm section shifts relative to the first rail arm section.

8. Orthotic according to claim 1, wherein the inclination adjustment system is designed such that the second rail arm section of at least one rail arm lies in a flexion position of the rail arm of up to 90° in a main plane of the articulated system or is arranged parallel thereto at least to a large extent, while the second rail arm section, when moving the rail arm from this flexion position in the direction of a 180° position, slopes increasingly in the medial or lateral direction.
